(19) United States
(12) Patent Application Publication
(10) Pub. No.: US 2023/0227786 A1
(43) Pub. Date: Jul. 20, 2023

(54) PREPARATION METHOD AND RECOVERY METHOD OF PARIDUVAL MESENCHYMAL STEM CELLS (PMSCs)

(71) Applicant: GUANGDONG VITALIFE BIOTECHNOLOGY CO., LTD, Foshan, Guangdong (CN)

(72) Inventors: Xiaocui Liu, Foshan, Guangdong (CN); Junrong Xu, Foshan, Guangdong (CN); Shuyan Tang, Foshan, Guangdong (CN); Yanlian Deng, Foshan, Guangdong (CN); Yanyao Meng, Foshan, Guangdong (CN); Jingli Yang, Foshan, Guangdong (CN); Jinhui Wang, Foshan, Guangdong (CN)

(21) Appl. No.: 18/009,473

(22) PCT Filed: Oct. 22, 2021
(86) PCT No.: PCT/CN2021/125480

§ 371 Date: Dec. 9, 2022

(30) Foreign Application Priority Data
Apr. 13, 2021 (CN) 202110392680.6

(51) Int. Cl. C12N 5/0775; A01N 1/02

(52) U.S. Cl. CPC C12N 5/0668; A01N 1/0221; A01N 1/0226; C12N 2500/90; C12N 2501/115; C12N 2500/32; C12N 2501/125; C12N 2501/11; C12N 2509/10

(57) ABSTRACT

The present disclosure discloses a preparation method and a recovery method of pariduval mesenchymal stem cells (PMSCs). In the preparation method, a high-glucose Dulbecco's Modified Eagle Medium (DMEM) that includes a Tryple-ethylenediaminetetraacetic acid (EDTA) enzyme of 40% to 60% in volume concentration and collagenase type II of 8 mg/ml to 12 mg/ml is used as a tissue digestion solution to digest tissue blocks, which facilitates PMSCs to climb out of the tissue blocks and grow adherently; and a serum-free DMEM is adopted as a selective medium to terminate the digestion and resuspend PMSCs, which helps to improve a purity of PMSCs, accelerate the growth of PMSCs, and achieve the rapid expansion of PMSCs in vitro.

The present application claims priority to Chinese Patent Application No. 202110392680.6 filed to the China National Intellectual Property Administration (CNIPA) on Apr. 13, 2021 and entitled “PREPARATION METHOD AND RECOVERY METHOD OF PARIDUVAL MESENCHYMAL STEM CELLS (PMSCs)”, which is incorporated herein by reference in its entirety.

### TECHNICAL FIELD

The present disclosure relates to the field of biotechnology, and in particular to a preparation method and a recovery method of pariduval mesenchymal stem cells (PMSCs).

### BACKGROUND

Mesenchymal stem cells (MSCs) are pluripotent progenitor cells with tumor chemotaxis, and the research on the application of MSCs in gynecologic oncology has made significant progress. However, there are different reports on the influence of uterus-derived MSCs on the biological behaviors of gynecological malignant tumor cells. The adaptability and selectivity of PMSCs for gynecological tumors are studied to develop a new method for clinical treatment of gynecological tumors.

PMSCs have the characteristics of simple enrichment cultivation in vitro, no violation of ethics, strong migration ability, homing to tumor cells, low immunogenicity, and the like. Since bone marrow-derived mesenchymal stem cells (BM-MSCs) are difficult to acquire and involve medical ethics, PMSCs can be used as a new carrier instead of BM-MSCs for tumor biotherapy. However, it is reported that maternal MSCs show different effects on the biological behaviors of malignant tumor cells, and thus the study on the adaptability and selectivity of PMSCs for various malignant tumors is a basis to determine whether PMSCs can be used as a carrier for malignant tumor treatment.

The existing PMSC acquisition method includes: separating a parietal decidua tissue and crushing it, and then cultivating the resulting cells adherently in a complete medium (Dulbecco's Modified Eagle Medium (DMEM) with 10% fetal bovine serum (FBS)) for 30 d. The conventional cryopreservation method for PMSCs includes: programmed cooling in a complete medium with 10% dimethyl sulfoxide (DMSO), and then cryopreservation in liquid nitrogen.

PMSCs are difficult to climb out of a tissue and grow adherently with the existing preparation method of PMSCs, and the existing medium cannot effectively promote the growth of PMSCs, such that the acquired cells are in a low concentration and have a poor quality, which compromises an inhibitory effect of PMSCs on tumor cells. In addition, the existing cryopreservation and recovery methods for PMSCs cannot effectively maintain the cell viability and biological function, and cannot guarantee that cells recovered in each batch show similar cell viabilities.

### SUMMARY

A technical problem to be solved by the present disclosure is to provide a preparation method of PMSCs. In the preparation method, cells are easy to climb out of a tissue and grow adherently, and a selective medium can effectively promote the growth of PMSCs, such that the acquired cells are in a high concentration and have a high quality, which can effectively inhibit tumor cells.

A technical problem to be solved by the present disclosure is to provide a recovery method of PMSCs, which can effectively maintain the cell viability and biological function, and ensure that cells recovered in each batch show similar cell viabilities.

A technical problem to be solved by the present disclosure is to provide a preparation method of PMSCs, including the following steps:

S11. cutting a parietal decidua tissue into tissue blocks of 1 mm³ to 4 mm³, and washing the tissue blocks with a tissue cleaning solution;

S12. subjecting the tissue blocks to digestion with a tissue digestion solution under constant temperature oscillation, terminating the digestion, filtering, and centrifuging to obtain a first precipitate, where the tissue digestion solution is a high-glucose DMEM that includes a Tryple-ethylenediaminetetraacetic acid (EDTA) enzyme of 40% to 60% in volume concentration and collagenase type II of 8 mg/ml to 12 mg/ml;

S13. washing the precipitate with normal saline (NS), resuspending, centrifuging, removing a first resulting supernatant, and resuspending with a selective medium to obtain a PMSC suspension;

S14. inoculating the PMSC suspension into a culture flask, conducting primary cell cultivation in an incubator, and denoting cells obtained as a P0 generation;

S15. when a cell confluency is greater than 80%, digesting, filtering, centrifuging, and resuspending a second precipitate with a selective medium for subculturing; and

S16. subjecting PMSCs of a Pn generation to digestion and centrifugation, discarding a second resulting supernatant, adding a cryopreservation solution to a resulting precipitate, and cryopreserving in a liquid nitrogen tank after programmed cooling, where n≥2.

As an improvement of the above solution, the selective medium may be a serum-free DMEM that includes a serum substitute of 8% to 12% in volume concentration, L-glutamine of 0.5 mol/ml to 1 mol/ml, a basic fibroblast growth factor (bFGF) of 18 ng/ml to 25 ng/ml, an epidermal growth factor (EGF) of 16 ng/ml to 22 ng/ml, and a stem cell growth factor (SCGF) of 6 ng/ml to 12 ng/ml.

As an improvement of the above solution, in S11, the tissue cleaning solution may be prepared from the following raw materials in volume percentage: 0.8% to 1.5% of a penicillin-streptomycin combination, 50% to 55% of an RBC lysis buffer, and 44% to 49% of NS; and the NS may have a mass fraction of 0.8% to 1%.

As an improvement of the above solution, in S12, the tissue blocks may be subjected to constant temperature oscillation in the tissue digestion solution for 1.5 h to 4 h at 36° C. to 39° C. and 150 rpm/min to 200 rpm/min;

the digestion may be terminated with a selective medium, and a volume of the selective medium may be 3 to 6 times a volume of the tissue digestion solution;

the filtering may be conducted using a filter screen with a pore size of 100 μm; and

the centrifuging may be conducted for 5 min to 7 min at a centrifugation speed of 1,200 rpm/min to 1,400 rpm/min.

As an improvement of the above solution, in S15, when the cell confluency is greater than 80%, a surface of the cells may be washed at least 2 times with phosphate-buffered saline (PBS);

the cells may be digested with a cell digestion solution for 3 min to 6 min, and then the digestion may be terminated with a selective medium;

a resulting mixture may be filtered through a filter screen with a pore size of 100 μm; and

a filtrate may be centrifuged for 5 min to 7 min at a centrifugation speed of 1,200 rpm/min to 1,400 rpm/min.

As an improvement of the above solution, the cell digestion solution may include trypsin of 0.1% to 0.15% in mass percentage and EDTA of 0.003% to 0.005% in mass percentage.

As an improvement of the above solution, before the PMSCs are collected, S16 may further include: subjecting the PMSCs to surface antibody marker assay, and only when positive indexes of CD73, CD90, and CD105 are each >99%, collecting the PMSCs.

As an improvement of the above solution, in S16, PMSCs of a P3 generation may be digested with trypsin and then centrifuged, the second resulting supernatant may be discarded, a cryopreservation solution may be added to the resulting precipitate, and a resulting mixture may be programed to cool down and cryopreserved in a liquid nitrogen tank.

As an improvement of the above solution, in S16, the cryopreservation solution may be a serum-free complete medium with Cryosure-DEX-40 of 18% to 25% in volume concentration; and

the cells may be cryopreserved at a density of 1.5×10⁶ to 2.5×10⁶ cells/ml.

Correspondingly, the present disclosure also provides a recovery method of PMSCs, including the following steps:

S21. thawing the cryopreserved PMSCs obtained above in a water bath at 36° C. to 39° C.; and

S22. resuspending the PMSCs obtained in S21 with a selective medium, centrifuging, and discarding a first resulting supernatant; washing a first resulting precipitate with PBS, centrifuging, and discarding a second resulting supernatant; and adding a selective medium to a second resulting precipitate, and transferring a resulting suspension to a culture flask for cultivation.

In some embodiments, the present disclosure provides a tissue digestion solution for the preparation of pariduval mesenchymal stem cells (PMSCs), wherein the tissue digestion solution is a high-glucose Dulbecco's Modified Eagle Medium (DMEM) that includes a Tryple-ethylenediaminetetraacetic acid (EDTA) enzyme of 40% to 60% in volume concentration and collagenase type II of 8 mg/ml to 12 mg/ml.

In some embodiments, the present disclosure provides a combination reagent for the preparation of PMSCs, including a tissue digestion solution and a selective medium, wherein the tissue digestion solution is a high-glucose DMEM that includes a Tryple-EDTA enzyme of 40% to 60% in volume concentration and collagenase type II of 8 mg/ml to 12 mg/ml; and the selective medium is a serum-free DMEM that includes a serum substitute of 8% to 12% in volume concentration, L-glutamine of 0.5 mol/ml to 1 mol/ml, a basic fibroblast growth factor (bFGF) of 18 ng/ml to 25 ng/ml, an epidermal growth factor (EGF) of 16 ng/ml to 22 ng/ml, and a stem cell growth factor (SCGF) of 6 ng/ml to 12 ng/ml.

Preferably, the combination reagent further includes a tissue cleaning solution, wherein the tissue cleaning solution is prepared from the following raw materials in volume percentage: 0.8% to 1.5% of a penicillin-streptomycin combination, 50% to 55% of a red blood cell (RBC) lysis buffer, and 44% to 49% of normal saline (NS); and the NS has a mass fraction of 0.8% to 1%.

Preferably, the combination reagent further includes a cell digestion solution, wherein the cell digestion solution includes trypsin of 0.1% to 0.15% in mass percentage and EDTA of 0.003% to 0.005% in mass percentage.

Preferably, the combination reagent further includes a cryopreservation solution, wherein the cryopreservation solution is a serum-free complete medium with Cryosure-DEX-40 of 18% to 25% in volume concentration.

In some embodiments, the present disclosure provides a method for inhibiting a proliferation ability of cancer cells, including using PMSCs prepared by the method above to inhibit the proliferation ability of the cancer cells, wherein the cancer cells include cervical cancer cells and/or breast cancer cells.

In some embodiments, the present disclosure provides a method for improving a viability of PMSCs, including using a feverfew extract to improve the viability of PMSCs, wherein a main active ingredient of the feverfew extract is parthenolide (PTL), and the feverfew extract includes one or more selected from the group consisting of a feverfew water extract, a feverfew alcohol extract, and a feverfew extract obtained from steam distillation.

The implementation of the present disclosure has the following beneficial effects:

1. The present disclosure adopts a high-glucose DMEM that includes a Tryple-EDTA enzyme of 40% to 60% in volume concentration and collagenase type II of 8 mg/ml to 12 mg/ml as a tissue digestion solution to digest tissue blocks, which facilitates PMSCs to climb out of the tissue blocks and grow adherently.

2. The present disclosure adopts a serum-free DMEM that includes a serum substitute of 8% to 12% in volume concentration, L-glutamine of 0.5 mol/ml to 1 mol/ml, a bFGF of 18 ng/ml to 25 ng/ml, an EGF of 16 ng/ml to 22 ng/ml, and an SCGF of 6 ng/ml to 12 ng/ml as a selective medium to terminate the digestion and resuspend PMSCs, which helps to improve a purity of PMSCs, accelerate the growth of PMSCs, and achieve the rapid expansion of PMSCs in vitro.

3. The recovery method of the present disclosure adopts a selective medium to recover PMSCs, such that the PMSCs can quickly recover and grow.

4. In the present disclosure, through the cooperation of a cryopreservation method and a recovery method, the cell viability and biological function of PMSCs can be effectively maintained, and it can be ensured that cells recovered in each batch show similar cell viabilities.

### BRIEF DESCRIPTION OF THE DRAWINGS

FIG. 1 is an electron microscopy image of P5 generation PMSCs cultivated in an upper chamber of a Transwell chamber for 3 d in Example 2 of the present disclosure;

FIG. 2 is an electron microscopy image of Hela cells cultivated and stabilized in a lower chamber of a Transwell chamber in Example 3 of the present disclosure;

FIG. 3 is an electron microscopy image of Hela cells co-cultivated with low-concentration PMSCs in Example 3 of the present disclosure;

FIG. 4 is an electron microscopy image of Hela cells co-cultivated with medium-concentration PMSCs in Example 3 of the present disclosure;

FIG. 5 is an electron microscopy image of Hela cells co-cultivated with high-concentration PMSCs in Example 3 of the present disclosure;

FIG. 6 shows the inhibition on the proliferation of Hela cells after the Hela cells are co-cultivated with PMSCs at different concentrations in Example 3 of the present disclosure;

FIG. 7 is an electron microscopy image of MCF-7 cells cultivated and stabilized in a lower chamber of a Transwell chamber in Example 4 of the present disclosure;

FIG. 8 is an electron microscopy image of low-concentration PMSCs and MCF-7 cells co-cultivated in Example 4 of the present disclosure;

FIG. 9 is an electron microscopy image of medium-concentration PMSCs and MCF-7 cells co-cultivated in Example 4 of the present disclosure;

FIG. 10 is an electron microscopy image of high-concentration PMSCs and MCF-7 cells co-cultivated in Example 4 of the present disclosure; and

FIG. 11 shows the inhibition on the proliferation of MCF-7 cells after the MCF-7 cells are co-cultivated with PMSCs at different concentrations in Example 4 of the present disclosure.

### DETAILED DESCRIPTION

In order to make the objectives, technical solutions, and advantages of the present disclosure more clear, the present disclosure will be further described in detail below with reference to the accompanying drawings. It should be noted that orientation terms such as “upper”, “lower”, “left”, “right”, “front”, “rear”, “inner”, and “outer” that appear or are about to appear in the present disclosure are only based on the accompanying drawings of the present disclosure, and do not specifically limit the present disclosure.

### Example 1

A preparation method of PMSCs was provided, including the following steps:

S11. collection of a parietal decidua tissue: a healthy term neonatal placental tissue was cleaned with a tissue cleaning solution to remove blood and blood clots, a parietal decidua tissue was peeled off using a surgical peeling instrument and then cut into tissue blocks of 1 mm³ to 4 mm³ using a surgical scissor, and the tissue blocks were cleaned with the tissue cleaning solution, where the tissue cleaning solution was prepared from the following raw materials in volume percentage: 1% of a penicillin-streptomycin combination, 51.1% of an RBC lysis buffer, and 47% of NS; and the NS had a mass fraction of 0.9%;

S12. digestion of the parietal decidua tissue blocks: a tissue digestion solution was added to the tissue blocks, and a resulting mixture was subjected to constant temperature oscillation for 2 h at 37° C. and 200 rpm/min, where the tissue digestion solution was a high-glucose DMEM including a Tryple-EDTA enzyme of 50% in volume concentration and collagenase type II of 10 mg/ml;

S13. termination of digestion: a selective medium was added at a volume 3 times a volume of the tissue digestion solution to terminate the digestion, a resulting mixture was filtered (with a 100 μm filter screen), and a filtrate was centrifuged at 1,300 rpm/min for 5 min to obtain a precipitate, where the selective medium was a serum-free DMEM including a serum substitute of 10% in volume concentration, L-glutamine of 0.8 mol/ml, a bFGF of 20 ng/ml, an EGF of 20 ng/ml, and an SCGF (Stem Cell Growth Factor) of 10 ng/ml;

S14. isolation of PMSCs: the precipitate was washed with NS and then resuspended to obtain a suspension, the suspension was centrifuged, a resulting supernatant was discarded, and a resulting precipitate was resuspended with a selective medium to obtain a PMSC suspension;

S15. cultivation of PMSCs: the obtained PMSC suspension was inoculated into a T75 culture flask, then the culture flask was statically placed in an incubator at 37° C., 5% CO₂, and saturated humidity for primary cell cultivation, and the obtained cells were denoted as a generation P0, where during the cultivation, a change in the medium was observed and the medium was changed every 2 d to 3 d;

S16. medium change and subculturing: after it was observed that a cell confluency was greater than 80%, a surface of adherent cells was washed with PBS to remove secretions, then the cells were digested with a cell digestion solution for 5 min, and the digestion was terminated with fresh complete medium; a resulting mixture was filtered through a 100 μm filter screen, a filtrate was centrifuged, and a resulting precipitate was resuspended with a cell medium; cells were counted, a survival rate was calculated, and the sterility and other indexes were tested; and the subculturing was conducted once every 2 d to 3 d, where the cell digestion solution included trypsin of 0.125% in mass fraction and EDTA of 0.004% in mass fraction;

S17. when the subculturing was conducted to P3, PMSC positive indexes were tested, and when positive indexes of CD73, CD90, and CD105 were each >99%, the subculturing was stopped, and cells were collected; and

S18. cryopreservation: trypsin was used to digest PMSCs of a P3 generation, a resulting mixture was centrifuged to obtain a precipitate, and a cryopreservation solution was added to the precipitate; the cells were counted and a survival rate was calculated; and the cells were programmed cooled and cryopreserved in a liquid nitrogen tank, where the cryopreservation solution was a serum-free complete medium with Cryosure-DEX-40 of 20% in volume concentration and the cells were cryopreserved at a density of 2×10⁶ cells/ml.

### Example 2

A recovery method of PMSCs was provided, including:

S21. the PMSCs cryopreserved in Example 1 were thawed in a water bath at 37° C.;

S22. the PMSCs obtained in S21 were resuspended with a selective medium, a resulting suspension was centrifuged at 1,300 rpm for 6 min, resulting cells were washed twice with PBS during which centrifugation was conducted at 1,300 rpm for 6 min, and a resulting precipitate was resuspended with a selective medium for subculturing; and

S23. PMSCs of a P5 generation were collected and resuspended with a selective medium to obtain a low-concentration group, a medium-concentration group, and a high-concentration group, each group was plated into an upper chamber of a Transwell chamber at a volume of 200 μL/well, and then the cells were cultivated for 3 d, where a concentration of PMSCs in the low-concentration group was 1×10⁵ cells/well, a concentration of PMSCs in the medium-concentration group was 2×10⁵ cells/well, and a concentration of PMSCs in the high-concentration group was 4×10⁵ cells/well. The morphology of PMSCs was shown in FIG. 1.

### Example 3

A co-cultivation test of cervical cancer cells with a PMSC medium was conducted, including:

S31. a cervical cancer cell line Hela was quickly thawed in a 37° C. water bath and then resuspended with a complete medium, a resulting suspension was centrifuged, and a resulting precipitate was washed twice with PBS during which centrifugation was conducted at 1,000 rpm for 3 min; and a resulting precipitate was resuspended with a fresh complete medium and transferred to a T25 culture flask for cultivation, which was denoted as a P1 generation, where the complete medium was a high-glucose serum-free DMEM including FBS of 10% in volume concentration and a penicillin-streptomycin combination of 1% in volume concentration;

S32. Hela cell subculturing: the morphology and medium change of recovered cells were observed, and the complete medium was changed every 2 d; when the cells grew to a cell confluency of greater than 80%, a cell suspension was collected; the cells were counted, a survival rate was calculated, microbial tests were conducted, and subculturing was conducted;

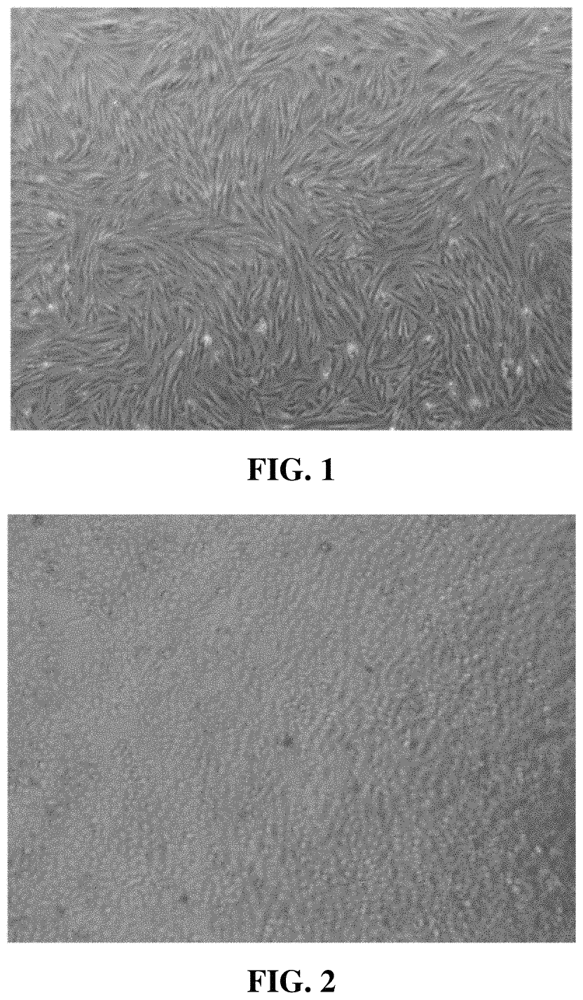

S33. cultivation of Hela cells in lower chambers of Transwell chambers: the original medium was discarded, the cells were washed twice with PBS and then digested for 2 min with 1 ml of trypsin, and then 10 ml of a complete medium was added to terminate the digestion; Hela cells of the P3 generation were collected, resuspended with a complete medium, and plated in a lower chamber of a Transwell chamber in each group of Example 2 at 2×10⁴ cells/well (24-well plate); and after the cells grew stably (12 h), the morphology of Hela cells was acquired, as shown in FIG. 2;

S34. co-cultivation: after the Hela cells in the lower chamber of the Transwell chamber grew stably, the upper chamber carrying PMSCs was placed into the well plate to allow co-cultivation with the Hela cells in the lower chamber for 3 d;

S35. growth of Hela cells: the growth of Hela cells was observed after co-cultivation for 3 d, and an image was acquired to record a growth state of the cells; and

S36. detection of the inhibition on proliferation: a blank group was set, and Hela cells in each group were collected after the co-cultivation to obtain a low-concentration co-cultivation group, a medium-concentration co-cultivation group, and a high-concentration co-cultivation group; and specifically, for each group, 5,000 cells were plated in each well of a 96-well plate and cultivated for 24 h, and then the proliferation was tested by a CCK-8 method.

Each experiment was repeated at least 3 times, and results were averaged.

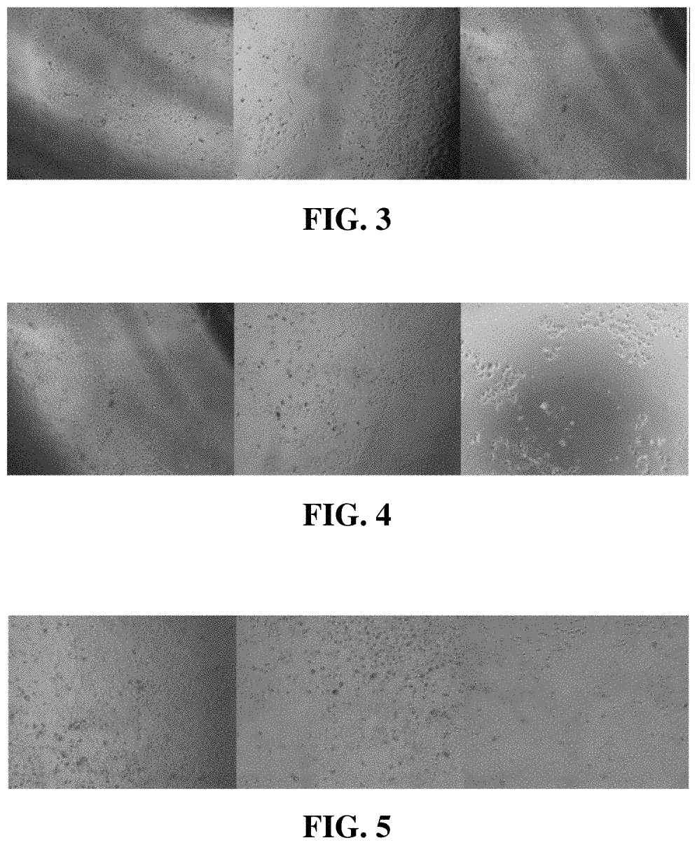

FIG. 3 is a cell morphology image of Hela cells co-cultivated with low-concentration PMSCs, FIG. 4 is a cell morphology image of Hela cells co-cultivated with medium-concentration PMSCs, and FIG. 5 is a cell morphology image of Hela cells co-cultivated with high-concentration PMSCs. FIG. 3 shows overlay images taken sequentially from left to right according to a distribution of Hela cells in the well plate (with an area occupied by the upper chamber of the chamber as a distribution center of Hela cells), where the left panel is a morphological image of Hela cells on an outer edge of the well plate after co-cultivation for 3 d in the low-concentration group, the middle panel is a morphological image of Hela cells in a transition circle of the well plate after co-cultivation for 3 d in the low-concentration group, and the right panel is a morphological image of Hela cells in a center of the well plate after co-cultivation for 3 d in the low-concentration group. FIG. 4 shows overlay images taken sequentially from left to right according to a distribution of Hela cells in the well plate (with an area occupied by the upper chamber of the chamber as a distribution center of Hela cells), where the left panel is a morphological image of Hela cells on an outer edge of the well plate after co-cultivation for 3 d in the medium-concentration group, the middle panel is a morphological image of Hela cells in a transition circle of the well plate after co-cultivation for 3 d in the medium-concentration group, and the right panel is a morphological image of Hela cells in a center of the well plate after co-cultivation for 3 d in the medium-concentration group. FIG. 5 shows overlay images taken sequentially from left to right according to a distribution of Hela cells in the well plate (with an area occupied by the upper chamber of the chamber as a distribution center of Hela cells), where the left panel is a morphological image of Hela cells on an outer edge of the well plate after co-cultivation for 3 d in the high-concentration group, the middle panel is a morphological image of Hela cells in a transition circle of the well plate after co-cultivation for 3 d in the high-concentration group, and the right panel is a morphological image of Hela cells in a center of the well plate after co-cultivation for 3 d in the high-concentration group.

It can be seen from FIG. 3 to FIG. 5 that the closer to the chamber carrying PMSCs, the sparser the Hela cells; and the Hela cells migrate from the outer edge to the center of the chamber. The cell morphological images in FIG. 3 to FIG. 5 obviously show that there are significant differences among different concentrations of PMSCs in the inhibition effect on the proliferation of the same Hela cells; and within a PMSC concentration range selected in this example, the higher the PMSC concentration, the more significant the inhibition on the proliferation of Hela cells.

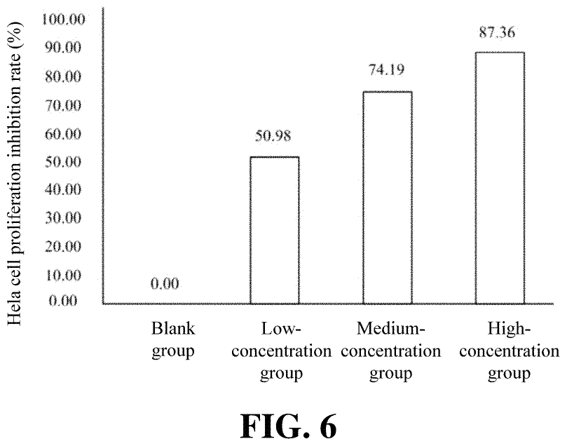

FIG. 6 shows the inhibition on the proliferation of Hela cells after the Hela cells were co-cultivated with PMSC media at different concentrations. Since no PMSC medium is added to co-cultivate with Hela cells in the blank group, an inhibition rate on the growth of Hela cells is zero in the blank group; and the higher the concentration of the PMSC medium, the higher the inhibition rate on the growth of Hela cells.

### Example 4

A co-cultivation test of breast cancer cells with a PMSC medium was conducted, including:

S31. a breast cancer cell line MCF-7 was quickly thawed in a 37° C. water bath and then resuspended with a complete medium, a resulting suspension was centrifuged, and a resulting precipitate was washed twice with PBS during which centrifugation was conducted at 1,000 rpm for 3 min; and a resulting precipitate was resuspended with a fresh complete medium and transferred to a T25 culture flask for cultivation, which was denoted as a P1 generation, where the complete medium was a high-glucose serum-free DMEM including FBS of 10% in volume concentration and a penicillin-streptomycin combination of 1% in volume concentration;

S32. MCF-7 cell subculturing: the morphology and medium change of recovered cells were observed, and the complete medium was changed every 2 d; when the cells grew to a cell confluency of greater than 80%, a cell suspension was collected; the cells were counted, a survival rate was calculated, microbial tests were conducted, and subculturing was conducted;

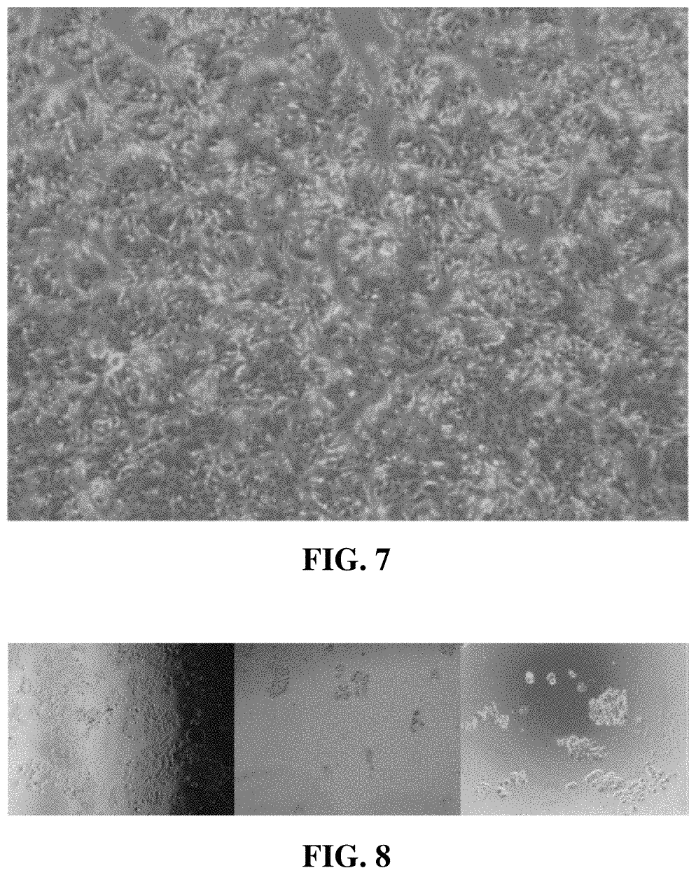

S33. cultivation of MCF-7 cells in lower chambers of Transwell chambers: the original medium was discarded, the cells were washed twice with PBS and then digested for 2 min with 1 ml of trypsin, and then 10 ml of a complete medium was added to terminate the digestion; MCF-7 cells of the P3 generation were collected, resuspended with a complete medium, and plated in a lower chamber of a Transwell chamber in each group of Example 2 at 2×10^{4 cells/well (}24-well plate); and after the cells grew stably (12 h), the morphology of MCF-7 cells was acquired, as shown in FIG. 7;

S34. co-cultivation: after the MCF-7 cells in the lower chamber of the Transwell chamber grew stably, the upper chamber carrying PMSCs was placed into the well plate to allow co-cultivation with the MCF-7 cells in the lower chamber for 3 d;

S35. growth of MCF-7 cells: the growth of MCF-7 cells was observed after co-cultivation for 3 d, and an image was acquired to record a growth state of the cells; and

S36. detection of the inhibition on proliferation: a blank group was set, and MCF-7 cells in each group were collected after the co-cultivation to obtain a low-concentration co-cultivation group, a medium-concentration co-cultivation group, and a high-concentration co-cultivation group; and specifically, for each group, 5,000 cells were plated in each well of a 96-well plate and cultivated for 24 h, and then the proliferation was tested by a CCK-8 method.

Each experiment was repeated at least 3 times, and results were averaged.

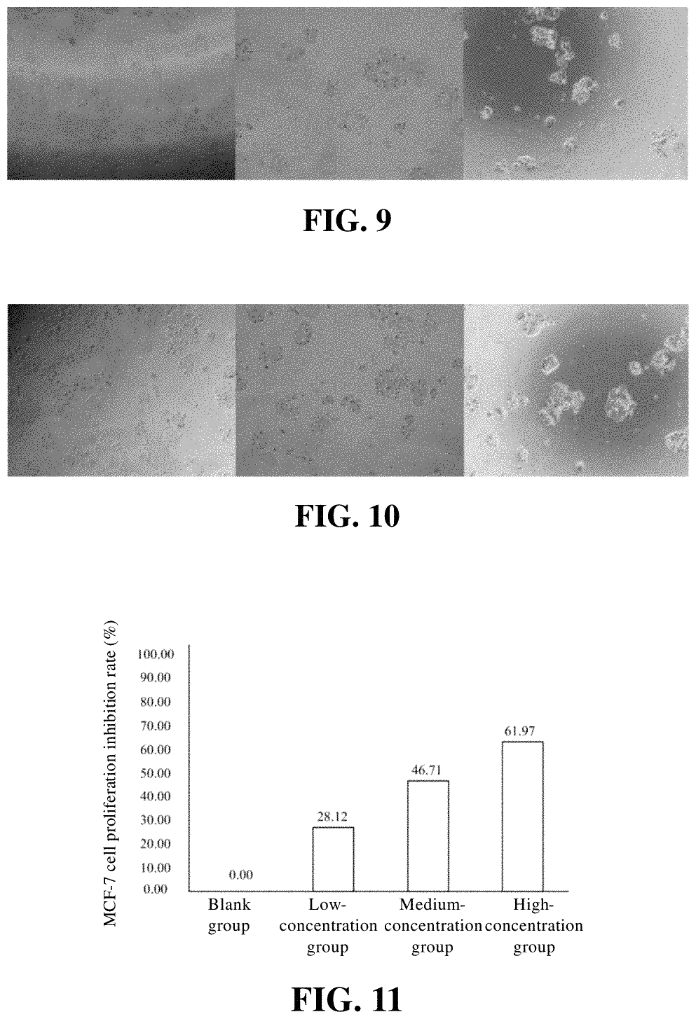

FIG. 8 is a cell morphology image of MCF-7 cells co-cultivated with low-concentration PMSCs, FIG. 9 is a cell morphology image of MCF-7 cells co-cultivated with medium-concentration PMSCs, and FIG. 10 is a cell morphology image of MCF-7 cells co-cultivated with high-concentration PMSCs. FIG. 8 shows overlay images taken sequentially from left to right according to a distribution of MCF-7 cells in the well plate (with an area occupied by the upper chamber of the chamber as a distribution center of cells), where the left panel is a morphological image of MCF-7 cells on an outer edge of the well plate after co-cultivation for 3 d in the low-concentration group, the middle panel is a morphological image of MCF-7 cells in a transition circle of the well plate after co-cultivation for 3 d in the low-concentration group, and the right panel is a morphological image of MCF-7 cells in a center of the well plate after co-cultivation for 3 d in the low-concentration group. FIG. 9 shows overlay images taken sequentially from left to right according to a distribution of MCF-7 cells in the well plate (with an area occupied by the upper chamber of the chamber as a distribution center of cells), where the left panel is a morphological image of MCF-7 cells on an outer edge of the well plate after co-cultivation for 3 d in the medium-concentration group, the middle panel is a morphological image of MCF-7 cells in a transition circle of the well plate after co-cultivation for 3 d in the medium-concentration group, and the right panel is a morphological image of MCF-7 cells in a center of the well plate after co-cultivation for 3 d in the medium-concentration group. FIG. 10 shows overlay images taken sequentially from left to right according to a distribution of MCF-7 cells in the well plate (with an area occupied by the upper chamber of the chamber as a distribution center of cells), where the left panel is a morphological image of MCF-7 cells on an outer edge of the well plate after co-cultivation for 3 d in the high-concentration group, the middle panel is a morphological image of MCF-7 cells in a transition circle of the well plate after co-cultivation for 3 d in the high-concentration group, and the right panel is a morphological image of MCF-7 cells in a center of the well plate after co-cultivation for 3 d in the high-concentration group.

It can be seen from FIG. 8 to FIG. 10 that the closer to the chamber carrying PMSCs, the sparser the MCF-7 cells; and the MCF-7 cells migrate from the outer edge to the center of the chamber. The cell morphological images in FIG. 8 to FIG. 10 obviously show that there are significant differences among different concentrations of PMSCs in the inhibition effect on the proliferation of the same MCF-7 cells; and within a PMSC concentration range selected in this example, the higher the PMSC concentration, the more significant the inhibition on the proliferation of MCF-7 cells.

FIG. 11 shows the inhibition on the proliferation of MCF-7 cells after the MCF-7 cells are co-cultivated with PMSC media at different concentrations. Since no PMSC medium is added to co-cultivate with MCF-7 cells in the blank group, an inhibition rate on the growth of MCF-7 cells is zero in the blank group; and the higher the concentration of the PMSC medium, the higher the inhibition rate on the growth of MCF-7 cells.

It can be seen from FIG. 6 and FIG. 11 that the PMSC medium has a significant inhibitory effect on the proliferation ability of both cervical cancer cells Hela and breast cancer cells MCF-7, where the inhibitory effect on cervical cancer cells Hela is significantly higher than the inhibitory effect on breast cancer cells MCF-7. It can be known that the PMSC medium shows adaptability and selectivity in the inhibitory effect on the proliferation ability of different gynecological tumors.

### Example 5

This example was different from Example 1 in that the selective medium was a serum-free DMEM including a serum substitute of 10% in volume concentration, L-glutamine of 0.8 mol/ml, a feverfew extract of 0.8 mg/ml, a bFGF of 20 ng/ml, an EGF of 20 ng/ml, and an SCGF of 10 ng/ml.

The feverfew extract was extracted by an existing extraction method (i.e. a steam distillation method), and a main active ingredient of the feverfew extract was parthenolide (PTL).

### Example 6

This example was different from Example 5 in that the selective medium was a serum-free DMEM including a serum substitute of 10% in volume concentration, L-glutamine of 0.8 mol/ml, a feverfew water extract of 0.8 mg/ml, a bFGF of 20 ng/ml, an EGF of 20 ng/ml, and an SCGF of 10 ng/ml.

A preparation method of the feverfew water extract was as follows:

S41. a feverfew was crushed to 100 mesh to obtain a feverfew powder; and

S42. the feverfew powder was added to deionized water, extraction was conducted at 75° C. for 90 min, and an extract was filtered out and dried to obtain the feverfew water extract, where a weight ratio of the feverfew powder to the deionized water was 1:9.

### Example 7

This example was different from Example 5 in that the selective medium was a serum-free DMEM including a serum substitute of 10% in volume concentration, L-glutamine of 0.8 mol/ml, a feverfew alcohol extract of 0.8 mg/ml, a bFGF of 20 ng/ml, an EGF of 20 ng/ml, and an SCGF of 10 ng/ml.

A preparation method of the feverfew alcohol extract was as follows:

S51. a feverfew was crushed to 100 mesh to obtain a feverfew powder; and

S52. the feverfew powder was added to a 60 wt % ethanol solution, extraction was conducted at 75° C. for 90 min, and an extract was filtered out and dried to obtain the feverfew alcohol extract, where a weight ratio of the feverfew powder to the ethanol solution was 1:9.

The allogeneic PMSCs prepared in Examples 1 and 5 and Comparative Examples 1 and 2 were stained with trypan blue and counted with a CountStar cell counter. A cell viability=number of viable cells/total number of cells×100%. Test results were shown in Table 1.

TABLE 1
Test results

| Group | Example 1 | Example 5 | Example 6 | Example 7 |
|---|---|---|---|---|
| Cell | 85 | 95.9 | 86.5 | 86.1 |
| viability (%) | | | | |

It can be seen from Table 1 that, the addition of the feverfew extract in Example 5 can significantly improve the cell viability. From the comparison of the feverfew water extract and the feverfew alcohol extract, it can be seen that the different feverfew extraction methods can significantly affect the improvement effect on the cell viability, and the feverfew extract obtained by the steam distillation method in Example 5 can significantly improve the cell viability.

The above examples are merely a part rather than all of the examples of the present disclosure. All other examples obtained by persons based on these examples without creative efforts shall fall within a protection scope of the present disclosure.

1. A tissue digestion solution for the preparation of pariduval mesenchymal stem cells (PMSCs), wherein the tissue digestion solution is a high-glucose Dulbecco's Modified Eagle Medium (DMEM) that comprises a Tryple-ethylenediaminetetraacetic acid (EDTA) enzyme of 40% to 60% in volume concentration and collagenase type II of 8 mg/ml to 12 mg/ml.

2. A combination reagent for the preparation of PMSCs, comprising the tissue digestion solution of claim 1 and a selective medium, wherein
the selective medium is a serum-free DMEM that comprises a serum substitute of 8% to 12% in volume concentration, L-glutamine of 0.5 mol/ml to 1 mol/ml, a basic fibroblast growth factor (bFGF) of 18 ng/ml to 25 ng/ml, an epidermal growth factor (EGF) of 16 ng/ml to 22 ng/ml, and a stem cell growth factor (SCGF) of 6 ng/ml to 12 ng/ml.

3. The combination reagent according to claim 2, further comprising a tissue cleaning solution, wherein the tissue cleaning solution is prepared from the following raw materials in volume percentage: 0.8% to 1.5% of a penicillin-streptomycin combination, 50% to 55% of a red blood cell (RBC) lysis buffer, and 44% to 49% of normal saline (NS); and the NS has a mass fraction of 0.8% to 1%.

4. The combination reagent according to claim 2, further comprising a cell digestion solution, wherein the cell digestion solution comprises trypsin of 0.1% to 0.15% in mass percentage and EDTA of 0.003% to 0.005% in mass percentage.

5. The combination reagent according to claim 2, further comprising a cryopreservation solution, wherein the cryopreservation solution is a serum-free complete medium with Cryosure-DEX-40 of 18% to 25% in volume concentration.

6. A preparation method of PMSCs, comprising the following steps:
S11. cutting a parietal decidua tissue into tissue blocks of 1 mm³ to 4 mm³, and washing the tissue blocks with a tissue cleaning solution;
S12. subjecting the tissue blocks to digestion with a tissue digestion solution under constant temperature oscillation, terminating the digestion, filtering, and centrifuging to obtain a first precipitate, wherein the tissue digestion solution is a high-glucose DMEM that comprises a Tryple-EDTA enzyme of 40% to 60% in volume concentration and collagenase type II of 8 mg/ml to 12 mg/ml;
S13. washing the precipitate with NS, resuspending, centrifuging, removing a first resulting supernatant, and resuspending with a selective medium to obtain a PMSC suspension;
S14. inoculating the PMSC suspension into a culture flask, conducting primary cell cultivation in an incubator, and denoting cells obtained as a P0 generation;
S15. when a cell confluency is greater than 80%, digesting, filtering, centrifuging, and resuspending a second precipitate with a selective medium for subculturing; and
S16. subjecting PMSCs of a Pn generation to digestion and centrifugation, discarding a second resulting supernatant, adding a cryopreservation solution to a resulting precipitate, and cryopreserving in a liquid nitrogen tank after programmed cooling, wherein n≥2.

7. The preparation method of PMSCs according to claim 6, wherein the selective medium is a serum-free DMEM that comprises a serum substitute of 8% to 12% in volume concentration, L-glutamine of 0.5 mol/ml to 1 mol/ml, a bFGF of 18 ng/ml to 25 ng/ml, an EGF of 16 ng/ml to 22 ng/ml, and an SCGF of 6 ng/ml to 12 ng/ml.

8. The preparation method of PMSCs according to claim 6, wherein in S11, the tissue cleaning solution is prepared from the following raw materials in volume percentage: 0.8% to 1.5% of a penicillin-streptomycin combination, 50% to 55% of an RBC lysis buffer, and 44% to 49% of NS; and the NS has a mass fraction of 0.8% to 1%.

9. The preparation method of PMSCs according to claim 6, wherein in S12, the tissue blocks are subjected to constant temperature oscillation in the tissue digestion solution for 1.5 h to 4 h at 36° C. to 39° C. and 150 rpm/min to 200 rpm/min;
the digestion is terminated with a selective medium, and a volume of the selective medium is 3 to 6 times a volume of the tissue digestion solution;
the filtering is conducted using a filter screen with a pore size of 100 μm; and
the centrifuging is conducted for 5 min to 7 min at a centrifugation speed of 1,200 rpm/min to 1,400 rpm/min.

10. The preparation method of PMSCs according to claim 6, wherein in S15, when the cell confluency is greater than 80%, a surface of the cells is washed at least 2 times with phosphate-buffered saline (PBS); and
the cells are digested with a cell digestion solution for 3 min to 6 min, and then the digestion is terminated with a selective medium.

11. The preparation method of PMSCs according to claim 6, wherein the cell digestion solution comprises trypsin of 0.1% to 0.15% in mass percentage and EDTA of 0.003% to 0.005% in mass percentage.

12. The preparation method of PMSCs according to claim 6, wherein before the PMSCs of the Pn generation are collected, S16 further comprises: subjecting the PMSCs of the Pn generation to surface antibody marker assay, and only when positive indexes of CD73, CD90, and CD105 are each >99%, collecting the PMSCs.

13. The preparation method of PMSCs according to claim 6, wherein in S16, PMSCs of a P3 generation are digested with trypsin and then centrifuged, the second resulting supernatant is discarded, a cryopreservation solution is added to the resulting precipitate, and a resulting mixture is programed to cool down and cryopreserved in a liquid nitrogen tank.

14. The preparation method of PMSCs according to claim 6, wherein in S16, the cryopreservation solution is a serum-free complete medium with Cryosure-DEX-40 of 18% to 25% in volume concentration; and
the cells are cryopreserved at a density of 1.5×10⁶ to 2.5×10⁶ cells/ml.

15. A recovery method of PMSCs, comprising the following steps:
S21. thawing the cryopreserved PMSCs obtained by the preparation method according to claim 6 in a water bath at 36° C. to 39° C.; and
S22. resuspending the PMSCs obtained in S21 with a selective medium, centrifuging, and discarding a first resulting supernatant; washing a first resulting precipitate with PBS, centrifuging, and discarding a second resulting supernatant; and adding a selective medium to a second resulting precipitate, and transferring a resulting suspension to a culture flask for cultivation.

16. A method for inhibiting a proliferation ability of cancer cells, comprising using PMSCs prepared by the method according to claim 6 to inhibit the proliferation ability of the cancer cells, wherein the cancer cells comprise cervical cancer cells and/or breast cancer cells.

17. A method for improving a viability of PMSCs, comprising using a feverfew extract to improve the viability of PMSCs, wherein a main active ingredient of the feverfew extract is parthenolide (PTL), and the feverfew extract comprises one or more selected from the group consisting of a feverfew water extract, a feverfew alcohol extract, and a feverfew extract obtained from steam distillation.

18. The combination reagent according to claim 3, further comprising a cell digestion solution, wherein the cell digestion solution comprises trypsin of 0.1% to 0.15% in mass percentage and EDTA of 0.003% to 0.005% in mass percentage.

19. The combination reagent according to claim 3, further comprising a cryopreservation solution, wherein the cryopreservation solution is a serum-free complete medium with Cryosure-DEX-40 of 18% to 25% in volume concentration.

20. The combination reagent according to claim 4, further comprising a cryopreservation solution, wherein the cryopreservation solution is a serum-free complete medium with Cryosure-DEX-40 of 18% to 25% in volume concentration.
